(19) United States
(12) Patent Application Publication
(10) Pub. No.: US 2008/0140172 A1
(43) Pub. Date: Jun. 12, 2008

(54) Multi-Wall Expandable Device Capable Of Drug Delivery Related Applications

(76) Inventors: Robert Hunt Carpenter, Bastrop, TX (US); Robert Owens Carpenter, Nashville, TN (US)

Correspondence Address: Robert Hunt Carpenter, 1303 Pecan Street, Bastrop, TX, 78602, US

(21) Appl. No.: 11/667,145

(22) PCT Filed: Dec. 9, 2005
(86) PCT No.: PCT/US2005/044692

§ 371 Date: May 5, 2007

Related U.S. Application Data
(60) Provisional application No. 60/635,590, filed on Dec. 13, 2004.
(60) Provisional application No. 60/645,842, filed on Jan. 21, 2005.

(51) Int. Cl. A61F 2/06

(52) U.S. Cl. 623/1.2; 623/1.28; 623/1.42

(57) ABSTRACT

A multi-wall expandable device (40) that optionally can have at least one material (46) between, Outside, or inside m the walls (43, 44) of the device. The sheet of material can be solid, perforated, or strands. Alternatively, individual strand or strands (47) of material may be woven through structural segments of the multi-wall expandable device. The sheet or sheets of material, strand, or strands can be used as a reservoir for any substance to be delivered, such as a drug, a supplement, a mineral or biological materials such as stem cells or a combination thereof. Additionally, the device is designed with the length of the device capable of being opened and closed to facilitate placement surrounding the treated structure to form a splint to offer support, protection, facilitate healing and deliver drugs or biological materials to the treated structure. This capability of being openable and closable can be accomplished with the use of latches, hinges, or memory metals such as nitinol.

### RELATED APPLICATIONS

This application claims priority to U.S. Provisional Patent Application, Ser. No. 60/645,842, entitled “MULTI-WALL EXPANDABLE DEVICE CAPABLE OF DRUG DELIVERY,” filed on Jan. 21, 2005, having Carpenter et al., listed as the inventors; and U.S. Provisional Patent Application, Ser. No. 60/635,590, entitled “MULTI-WALL DEVICE CAPABLE OF DRUG DELIVERY,” filed on Dec. 13, 2004, having Carpenter et al., listed as the inventors, the entire content of each is hereby incorporated by reference.

### STATEMENT OF RIGHTS TO INVENTIONS MADE UNDER FEDERALLY SPONSORED RESEARCH

No federal grants or funds were used in the development of the present invention.

### BACKGROUND

The present invention relates to a multi-wall expandable device that optionally can have at least one sheet of material between, outside, or inside the walls of the device. The sheet of material can be solid, perforated, or strands. Alternatively, individual strand or strands of material may be woven through structural segments of the multi-wall expandable device. For the purposes of this application, the word expandable means both expandable in dimensions, or; closable, or openable. The device is self-aligning, expandable, or can be caused to have variable diameter that may allow the device to be opened, or removed after implantation. More specifically, the present invention relates to a multi-wall expandable device, such as a multi-wall stent or splint, that can be used for intravascular diameter maintenance, or outer surface support, to protect, maintain patency, deliver substances to, or facilitate repair of biological structures. The sheet or sheets of material, strand, or strands can be used as a reservoir for any substance to be delivered, such as a drug, a supplement, mineral, biological materials such as stem cells or a combination thereof. The multi-wall expandable device may have multiple walls only at one end, or both ends, where the multiple walls do not encompass, or extends to, the entire length of the multi-wall expandable device. Additionally, the device is designed with the length of the device capable of being opened to facilitate placement surrounding the treated structure to form a splint to offer protection, facilitate healing, or deliver drugs or biological materials to the treated structure. This capability of being openable, or closable can be accomplished with the use of latches, hinges, or memory metals such as nitinol. The latches can be mechanical, magnetic or any other method by which the two halves of the device can be secured together. The hinge may be mechanical with hinge-pins, a curved memory hinge, or a nano-scale ductile hinge. The combination of hinges, or latches can be optional, or variable depending on the size, or use of the device.

Life is based on the maintenance of biological structures. Modern biology deals primarily with the structure, or function of biological structures. The primary challenge of modern medicine is dealing with conditions caused by the loss of integrity of biological structures. While it is relatively easy to deal with loss of integrity in thick walled biological structures, medical intervention in thin wall, or delicate biological structures such as nerves is very complicated. Complex biological structures such as those found in the nervous system, lymphatic system, or the circulatory system present unique challenges for medical intervention to prevent or correct stenosis, deliver therapeutic compounds, or maintain patency.

There are a large number of implantable devices, or instruments that have been used in interventional medicine to treat loss of integrity, stenosis, or maintain patency of biological structures. These devices all have a primary disadvantage of not being retrievable. Recently, drug coated devices have been used with mixed results to prevent restenosis, or enhance healing. While the drug coated devices are improvements over previous devices, the amount of drug delivered, or uniform dose to the target tissue over a long period of time are problematic. After the drug is depleted from the device, restenosis often occurs, or one is left with a useless device that can only be removed through surgery.

U.S. Pat. No. 5,667,523 describes a dual supported intraluminal graft having a biocompatible flexible layer sandwiched between two structural support layers. The two structural support layers are concentrically positioned with respect to one another.

Current devices may not be self aligning following placement or implantation. The ability for the device to accommodate changes in surrounding tissues due to healing or reduction in swelling in the local area may be important for long periods after implantation. Current technologies are specifically not suited for use in delicate structures such as nerve tissues or other biological structures. The current devices are not flexible enough, or have relatively sharp ends that may result in erosion or perforation of the implanted biological structure. Further, current splints are almost impossible to remove once the device has been inserted into a biological structure.

Thus, there is a need for a device that can overcome all the problems associated with currently available devices.

### SUMMARY

The present invention relates to a multi-wall device that optionally can have at least one sheet of material between, outside, or inside the walls of the device. The sheet of material can be solid, perforated or a strand. The multi-wall expandable device having a plurality of radially expandable tubular members, each tubular member axially extending along a longitudinal axis of the multi-wall expandable device, and each tubular member having first and second opposing ends, wherein adjacent tubular members are connected at least at one end to form a seamless joint. The tubular members can be solid materials, perforated materials, strands, or combination thereof. Alternatively, individual strand or strands of material may be woven through structural segments of the multi-wall expandable device. For the purposes of this application, the word expandable means both expandable in dimensions, or; closable, or openable. The device can be caused to have variable diameter that may allow the device to be opened, or removed after implantation. Further, the multi-wall expandable device of the present invention can be self-aligning when placed next to outside surfaces a tubular structure. More specifically, the present invention relates to a device, such as a stent or splint, that can be used to protect, maintain patency, deliver substances to, or facilitate repair of biological structures. The sheet of material or sheets of materials, strand, or strands can be used as a reservoir for a substance to be delivered, such as a drug, a supplement, mineral, biological materials such as stem cells or a combination thereof. The multi-wall expandable device may have multiple walls only at one end or at both ends of the multi-wall device, where the multiple walls do not extend to the entire length of the multi-wall device. Additionally, the device is designed with the length of the device capable of being opened, or closed to facilitate placement surrounding the treated structure to form a splint to offer protection, facilitate healing, or deliver drugs or biological materials to the treated structure. This capability of being openable, or closable can be accomplished with the use of latches, hinges, or memory metals such as nitinol. The latches can be mechanical, magnetic or any other method by which the two halves of the device can be secured together. The hinge may be mechanical with hinge-pins, a curved memory hinge, or a nano-scale ductile hinge. The combination of hinges, or latches can be optional, or variable depending on the size, or use of the device.

One aspect of the present invention relates to a multi-wall expandable device capable of being opened, or closed along its length to facilitate placement to form a splint surrounding a structure to be treated. The walls of the multi-wall expandable device can be made from a perforated or mesh material. The multi-wall expandable device has a first end, or a second end, having a hollow passage path running along the length of the expandable device. The walls of the multi-wall expandable device can be permanently joined with a seam at the first end, or the second end, or both. The walls of the multi-wall expandable device can also be continuous without a seam, i.e. seamless, at the first end, or the second end, or both. Alternatively, one end has a seam permanently joining the multiple walls, or the multiple walls are joined in a seamless way at the other end. Alternatively, a combination of seam, or seamless joining of the multiple walls can be used at either end or at both ends. Additionally, the device is designed with the length of the device capable of being opened, or closed to facilitate placement surrounding the treated structure to form a splint to offer protection, facilitate healing, or deliver drugs or biological materials to the treated structure. This capability of being openable, or closable can be accomplished with the use of latches, hinges, or memory metals such as nitinol. The latches can be mechanical, magnetic or any other method by which the two halves of the device can be secured together. The hinge may be mechanical with hinge-pins, a curved memory hinge, or a nano-scale ductile hinge. The combination of hinges, or latches can be optional, or variable depending on the size, or use of the device.

Other embodiments of the present invention relates to multi-wall devices in which the multiple walls do not extend all the way from one end of the devices to the other end of the devices. Here, only one end, or both ends, of the devices would have the multilayer walls. At this end, or at both ends, the multiple walls can be joined in a seamless fashion, like a fold, or joined with seam or seams, or a combination of the seamless fashion together with seam or seams. Preferably, the multi-wall expandable device that does not have multiple walls extending the entire length of the device would have the multiple walls joined in a seamless fashion, like a fold or folds. Preferably, the “fold” or “folds” are folded outwardly.

The film, films, strand, or strands can be interposed, intertwined, or wrapped around spaces created by the multiple walls, around the outside or inside of the multi-wall expandable device of the present invention. The film can be porous or solid.

One embodiment of the present invention relates to a device that incorporates a double-wall tubular device that may be: (1) formed by two tubular structures placed one inside the other with or without a seam or seams at either or both ends (FIG. 1A), (2) woven to be a seamless double wall tube at both ends (FIG. 2A), (3) woven to be a seamless double wall tube on one end and a seam on the other end (FIG. 3A); or (4) a double wall tube on the first end and the second end connected by a single wall middle segment (FIG. 4A).

Additionally, the device can be removed with a special catheter equipped with a guarded treble hook canula. The structure of the device causes it to reduce its diameter when the hooks are engaged and a pulling and twisting pressure are applied. Removal is facilitated by pulling the device into the catheter that has been advanced to the first end of the device. Then the guarded treble hook canula is advanced out the end of the catheter and the hooks are engaged in the inner wall structural segments near the second end of the device. The device is then drawn slowly and carefully into the catheter and the catheter removed from the tubular structure.

Another embodiment of the present invention relates to a device that incorporates a multiple-wall expandable device that may be: (1) formed by two tubular structures placed one inside the other with or without a seam or seams at either or both ends (FIG. 5A), (2) woven to be a seamless double wall tube at both ends (FIG. 6A), (3) woven to be a seamless double wall tube on one end and a seam on the other end (FIG. 7A); or (4) a double wall tube on the first end and the second end connected by a single wall middle segment (FIG. 8A). Any of these embodiments may be formed with the length of the device capable of being opened and closed to facilitate the placement to the device to form a splint surrounding a biological structure. This capability of being openable and closable can be accomplished with the use of latches, hinges, or memory metals such as nitinol. The latches can be mechanical, magnetic or any other method by which the two halves of the device can be secured together. The hinge may be mechanical with hinge-pins, a curved memory hinge, or a nano-scale ductile hinge. The combination of hinges and latches can be optional, or variable depending on the size, or use of the device. (FIGS. 5A-8A).

This double-wall construction facilitates the easy incorporation of other useful materials between the two walls. These materials can deliver drug substances, supplements, minerals, or biological materials such as stem cells or combinations thereof. Additionally, these materials can prevent adhesion, facilitate flow through the device and/or facilitate the device recovery.

Additionally, the device can be delivered and removed with a special instrument that allows the device to be secured, or opened, or closed along its long axis, or placed surrounding the structure to be treated.

A unique application of this device is the delivery of stem cells in a culture matrix via the specialized instrument to tissues to be implanted and revitalized. A double wall device with a matrix between the walls suitable for the adherence of cells will be placed in a culture of stem cells. As these cells colonize the matrix, they will develop connections between adjoining cells on the matrix. When the device is colonized and the stem cells are stable, the device is then transferred to the implantation site via the special instrument, or the device is allowed to remain at the implantation site for a few days. During this time the cells will migrate from the device matrix and colonize the adjoining tissues. Additionally they will maintain their connections established in culture to the adjoining cells as they migrate into the tissues. This capability is especially important if the stem cells are being used to repair cardiac tissues, nerve tissues or any other tissue where cell-cell communications are important. The stem cell transport device can then be removed if required.

### BRIEF DESCRIPTION OF THE DRAWINGS

FIG. 1A shows a double-wall device, 30, with each of the first end and the second end that may be permanently joined at a seam;

FIG. 1B shows a double-wall device with each of the first end and the second end that may be permanently joined at a seam, having one solid sheet of material sandwiched between the outer and the inner wall of the double-wall device;

FIG. 1C shows a double-wall device with each of the first end and the second end that may be permanently joined at a seam, having one or more strands of material sandwiched between the outer and the inner wall of the double-wall device;

FIG. 2A shows a double-wall device, 40, with each of the first end and the second end being continuous, seamless, but folded;

FIG. 2B shows a double-wall device with each of the first end and the second end being continuous, seamless, but folded, having one solid sheet of material sandwiched between the outer and the inner wall of the double-wall device;

FIG. 2C shows a double-wall device with each of the first end and the second end being continuous, seamless, but folded, having one or more strands of material sandwiched between the outer and the inner wall of the double-wall device;

FIG. 3A shows a double-wall device, 50, with the first end being continuous, seamless, but folded and the second end that may be permanently joined at a seam;

FIG. 3B shows a double-wall device with the first end being continuous, seamless, but folded and the second end that may be permanently joined at a seam, having one solid sheet of material sandwiched between the outer and the inner wall of the double-wall device;

FIG. 3C shows a double-wall device with the first end being continuous, seamless, but folded and the second end that may be permanently joined at a seam, having one or more strands material sandwiched between the outer and the inner wall of the double-wall device;

FIG. 4A shows a double-wall device, 60, with both the first end and the second end having double walls, and the middle segment connecting the double wall ends is single wall;

FIG. 4B shows a double-wall device with both the first end and the second end having double walls, and the single wall middle segment connecting the double wall ends is covered with a sheet of suitable material;

FIG. 4C shows a double-wall device with both the first end and the second end having double walls, with the ends having one or more strands of material sandwiched between the outer and the inner wall of the double-wall ends of the device, while the middle segment has a single wall;

FIG. 4D shows a double-wall device with both the first end and the second end having double walls, with the ends having one or more strands of material sandwiched between the outer and the inner wall of the double-wall ends of the device and a different strand surrounding the single wall middle segment;

FIG. 4E shows a double-wall device with both the first end and the second end having double walls, with the ends having one or more strands of material sandwiched between the outer and the inner wall of the double-wall ends of the device and a sheet of material surrounding the single wall middle segment;

FIG. 4F shows a double-wall device with both the first end and the second end having double walls, with one of the ends having one or more strands of material sandwiched between the outer and the inner wall of the double-wall ends of the device and the other end containing a sheet of material in a similar manner and a sheet of material surrounding the single wall middle segment.

In the above drawings, the hexagonal structural segments drawn in solid lines indicate the outer wall, while the hexagonal structural segments drawn in the broken lines indicate the inner wall. Bolded solid lines indicate strands in the outer structural segments and bolded broken lines indicate strands in the inner structural segments. A broad layer of dots indicated a sheet of material.

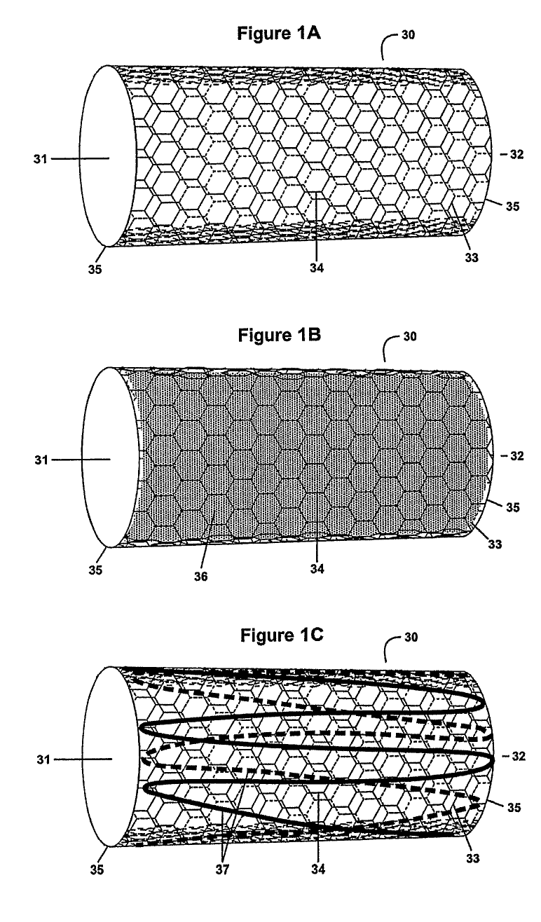

In FIGS. 1A-1C: 31 is the first end opening, 32 is the second end opening, 33 is the inner wall structural segments, 34 is the outer wall structural segments, 35 is the joining of the inner and outer wall, 36 is the sheet of material sandwiched between the walls, and 37 is the strands of material in or between the walls.

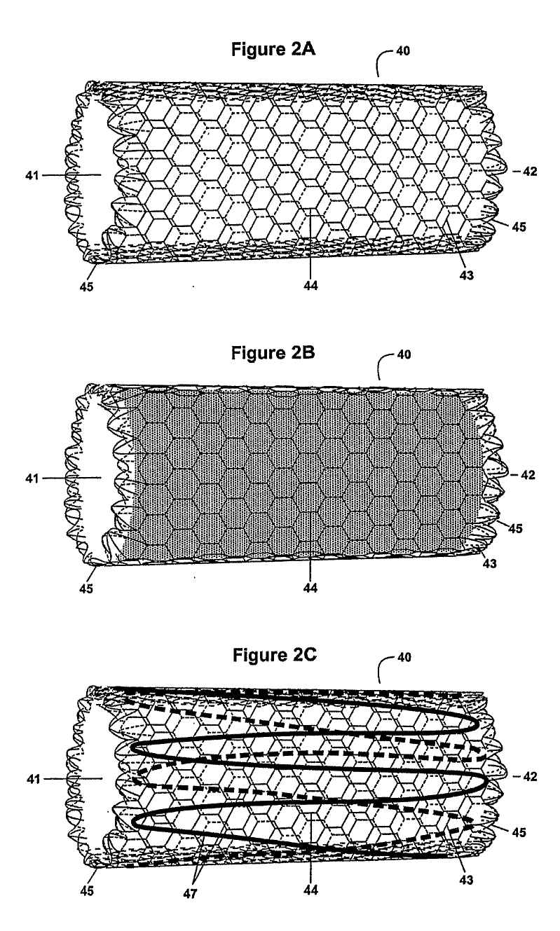

In FIGS. 2A-2C: 41 is the first end opening, 42 is the second end opening, 43 is the inner wall structural segments, 44 is the outer wall structural segments, 45 is the joining of the inner and outer wall, 46 is the sheet of material sandwiched between the walls, and 47 is the strands of material in or between the walls.

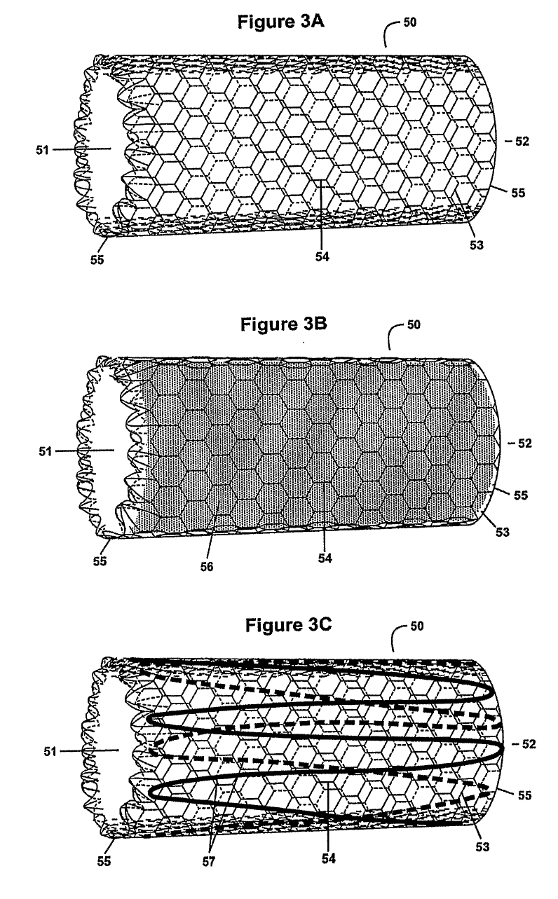

In FIGS. 3A-3C: 51 is the first end opening, 52 is the second end opening, 53 is the inner wall structural segments, 54 is the outer wall structural segments, 55 is the joining of the inner and outer wall, 56 is the sheet of material sandwiched between the walls, and 57 is the strands of material in or between the walls.

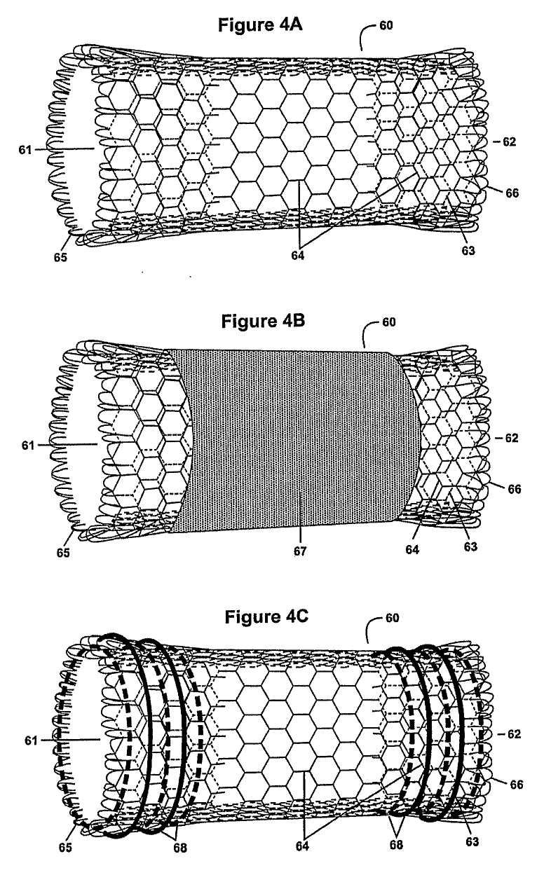

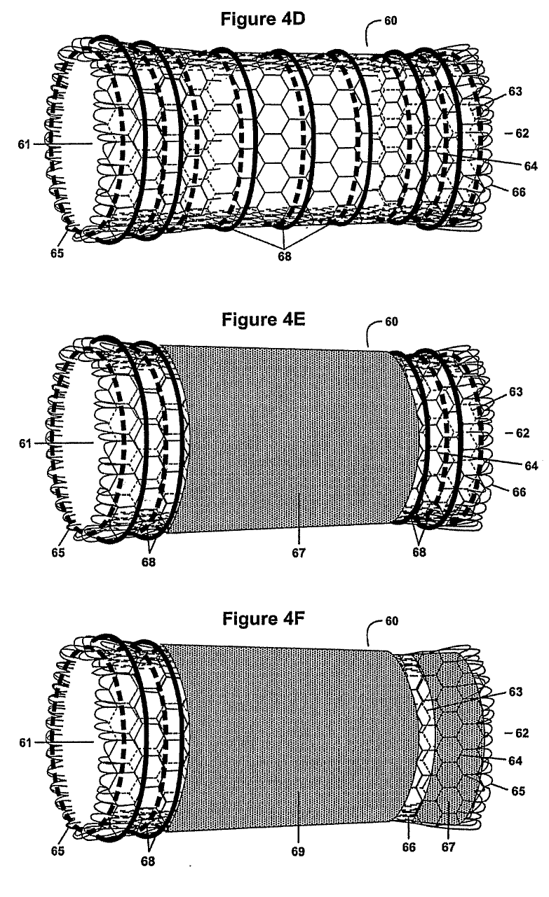

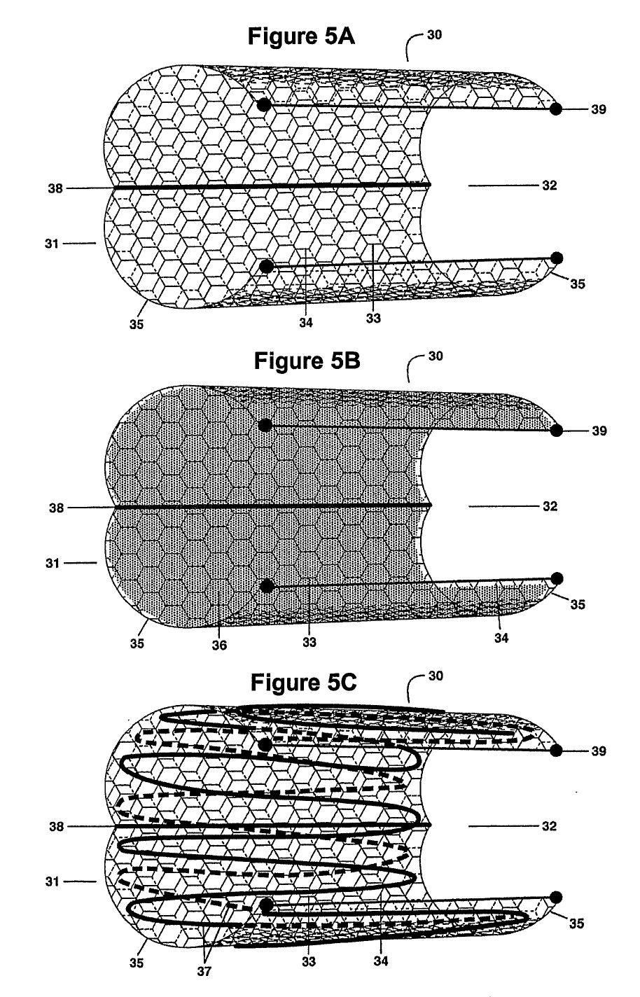

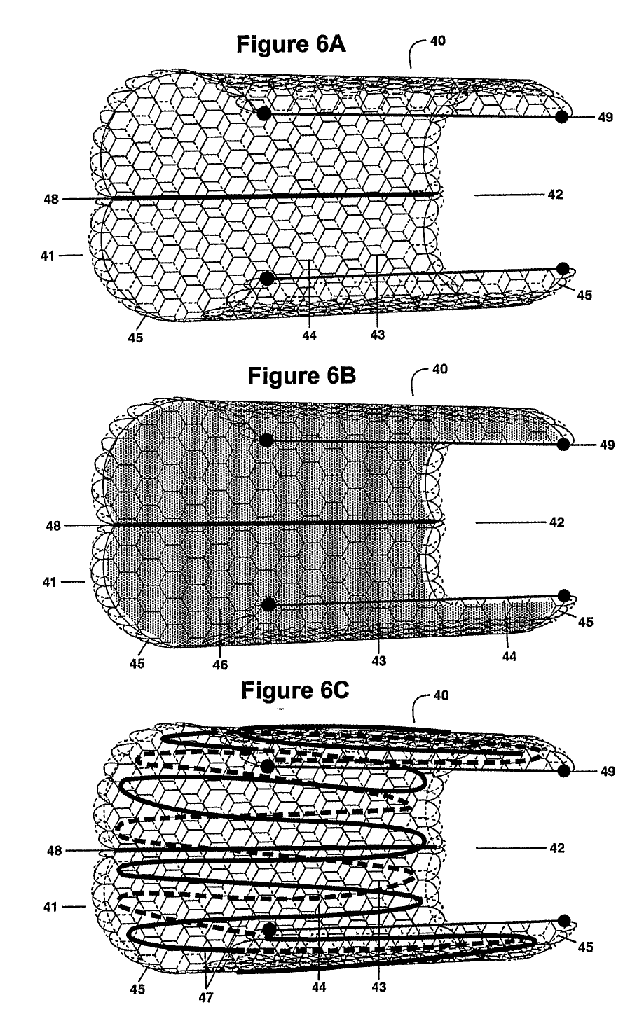

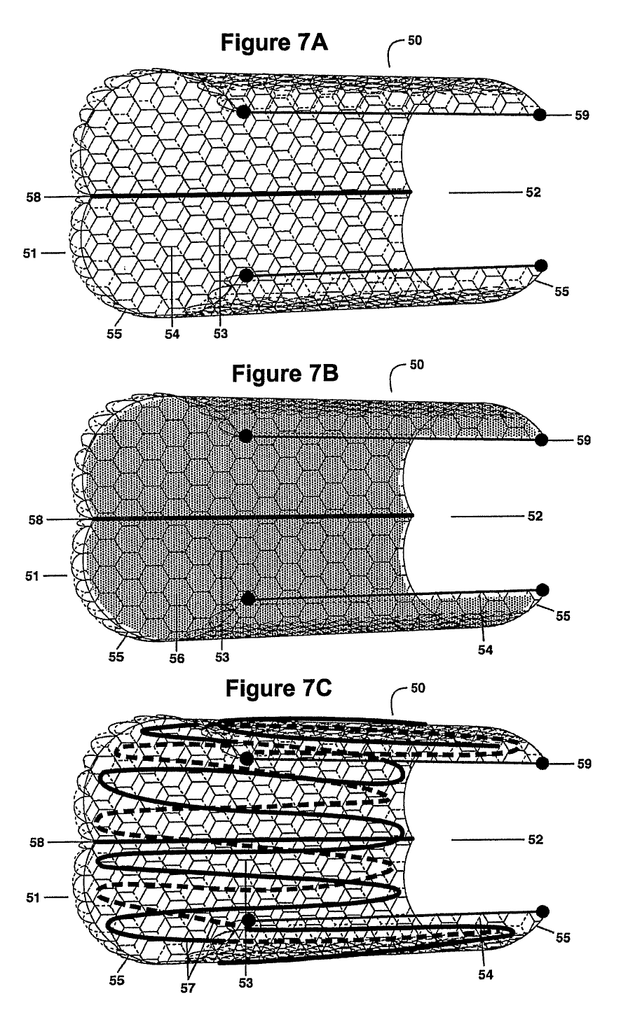

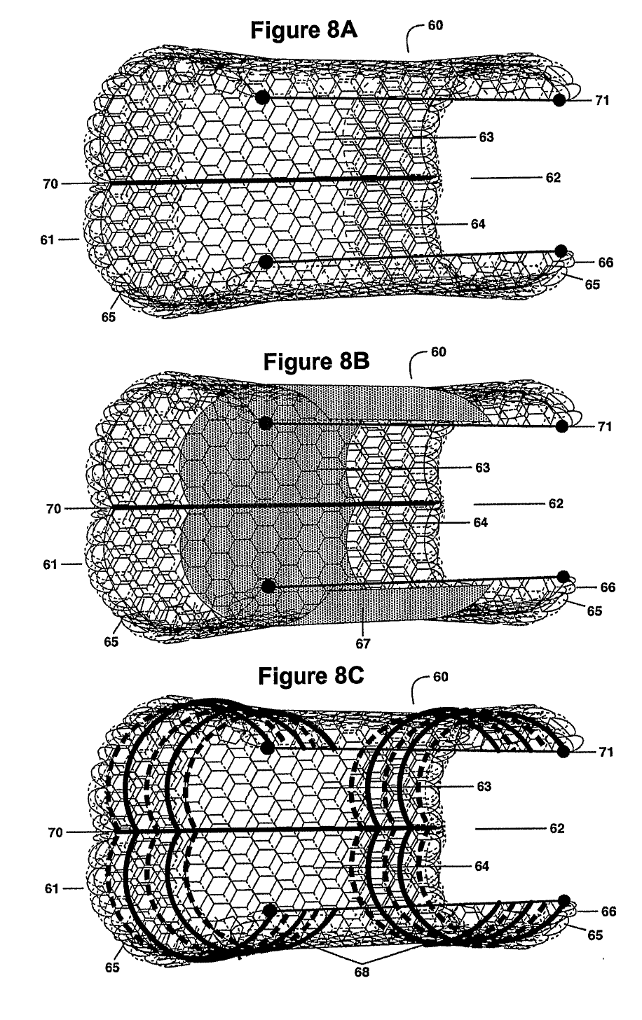

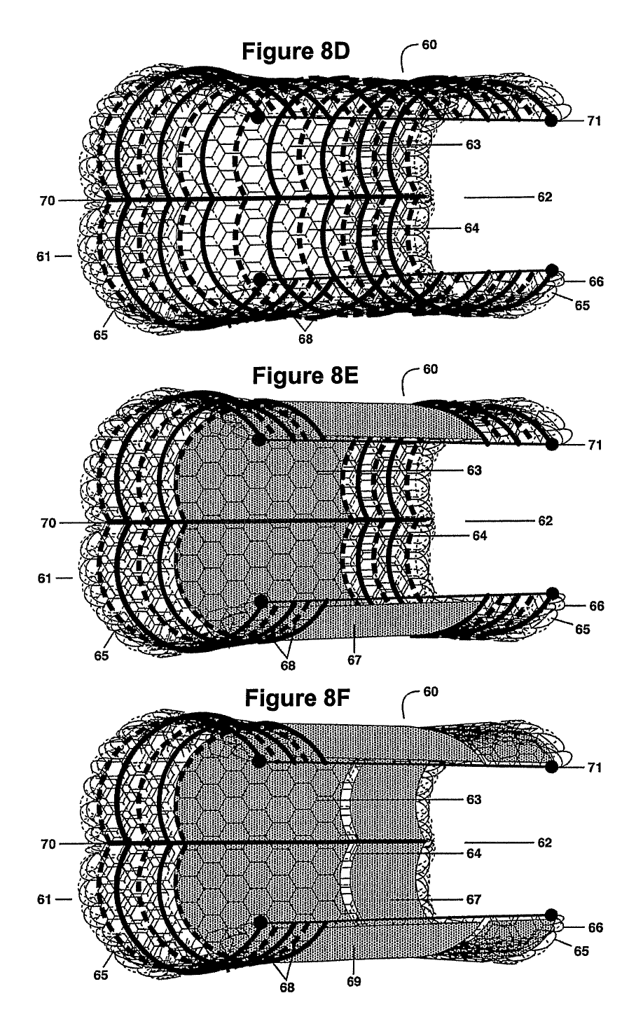

In FIGS. 4A-4F: 61 is the first end opening, 62 is the second end opening, 63 is the inner wall structural segments, 64 is the outer wall structural segments, 65 is the joining of the inner and outer wall on the first end, 66 is the joining of the inner and outer wall on the second end, 67 is the sheet of material sandwiched between the walls, and 68 is the strands of material in or between the walls, 69 is the sheet of material surrounding the single wall of the device.

FIG. 5A shows a double-wall device, 30, with each of the first end, 31, and the second end, 32, that may be permanently joined at a seam. This device has a longitudinal opening with optional hinges on one side, 38, or latches, 39, on the other to facilitate the placement surrounding a biological or other physical structure and to facilitate the removal of the device;

FIG. 5B shows a double-wall device, 30, with each of the first end and the second end that may be permanently joined at a seam, having one solid sheet of material sandwiched between the outer and the inner wall of the double-wall device. This device has a longitudinal opening with optional hinges on one side, 38, and latches, 39, on the other to facilitate the placement surrounding a biological or other physical structure and to facilitate the removal of the device;

FIG. 5C shows a double-wall device, 30, with each of the first end and the second end that may be permanently joined at a seam, having one or more strands of material sandwiched between the outer and the inner wall of the double-wall device. This device has a longitudinal opening with optional hinges on one side, 38, and latches, 39, on the other to facilitate the placement surrounding a biological or other physical structure and to facilitate the removal of the device;

FIG. 6A shows a double-wall device, 40, with each of the first end and the second end being continuous, seamless, but folded. This device has a longitudinal opening with optional hinges on one side, 48, and latches, 49, on the other to facilitate the placement surrounding a biological or other physical structure and to facilitate the removal of the device;

FIG. 6B shows a double-wall device, 40, with each of the first end and the second end being continuous, seamless, but folded, having one solid sheet of material sandwiched between the outer and the inner wall of the double-wall device. This device has a longitudinal opening with optional hinges on one side, 48, and latches, 49, on the other to facilitate the placement surrounding a biological or other physical structure and to facilitate the removal of the device;

FIG. 6C shows a double-wall device, 40, with each of the first end and the second end being continuous, seamless, but folded, having one or more strands of material sandwiched between the outer and the inner wall of the double-wall device. This device has a longitudinal opening with optional hinges on one side, 48, and latches, 49, on the other to facilitate the placement surrounding a biological or other physical structure and to facilitate the removal of the device;

FIG. 7A shows a double-wall device, 50, with the first end being continuous, seamless, but folded and the second end that may be permanently joined at a seam. This device has a longitudinal opening with optional hinges on one side, 58, and latches, 59, on the other to facilitate the placement surrounding a biological or other physical structure and to facilitate the removal of the device;

FIG. 7B shows a double-wall device, 50, with the first end being continuous, seamless, but folded and the second end that may be permanently joined at a seam, having one solid sheet of material sandwiched between the outer and the inner wall of the double-wall device. This device has a longitudinal opening with optional hinges on one side, 58, and latches, 59, on the other to facilitate the placement surrounding a biological or other physical structure and to facilitate the removal of the device;

FIG. 7C shows a double-wall device, 50, with the first end being continuous, seamless, but folded and the second end that may be permanently joined at a seam, having one or more strands material sandwiched between the outer and the inner wall of the double-wall device. This device has a longitudinal opening with optional hinges on one side, 58, and latches, 59, on the other to facilitate the placement surrounding a biological or other physical structure and to facilitate the removal of the device;

FIG. 8A shows a double-wall device, 60, with both the first end and the second end having double walls, and the middle segment connecting the double wall ends is single wall. This device has a longitudinal opening with optional hinges on one side, 70, and latches, 71, on the other to facilitate the placement surrounding a biological or other physical structure and to facilitate the removal of the device;

FIG. 8B shows a double-wall device, 60, with both the first end and the second end having double walls, and the single wall middle segment connecting the double wall ends is covered with a sheet of suitable material. This device has a longitudinal opening with optional hinges on one side, 70, and latches, 71, on the other to facilitate the placement surrounding a biological or other physical structure and to facilitate the removal of the device;

FIG. 8C shows a double-wall device, 60, with both the first end and the second end having double walls, with the ends having one or more strands of material sandwiched between the outer and the inner wall of the double-wall ends of the device, while the middle segment has a single wall. This device has a longitudinal opening with optional hinges on one side, 70, and latches, 71, on the other to facilitate the placement surrounding a biological or other physical structure and to facilitate the removal of the device;

FIG. 8D shows a double-wall device, 60, with both the first end and the second end having double walls, with the ends having one or more strands of material sandwiched between the outer and the inner wall of the double-wall ends of the device and a different strand surrounding the single wall middle segment. This device has a longitudinal opening with optional hinges on one side, 70, and latches, 71, on the other to facilitate the placement surrounding a biological or other physical structure and to facilitate the removal of the device;

FIG. 8E shows a double-wall device, 60, with both the first end and the second end having double walls, with the ends having one or more strands of material sandwiched between the outer and the inner wall of the double-wall ends of the device and a sheet of material surrounding the single wall middle segment. This device has a longitudinal opening with optional hinges on one side, 70, and latches, 71, on the other to facilitate the placement surrounding a biological or other physical structure and to facilitate the removal of the device;

FIG. 8F shows a double-wall device, 60, with both the first end and the second end having double walls, with one of the ends having one or more strands of material sandwiched between the outer and the inner wall of the double-wall ends of the device and the other end containing a sheet of material in a similar manner and a sheet of material surrounding the single wall middle segment. This device has a longitudinal opening with optional hinges on one side, 70, and latches, 71, on the other to facilitate the placement surrounding a biological or other physical structure and to facilitate the removal of the device; and

In the above drawings, the hexagonal structural segments drawn in solid lines indicate the outer wall, while the hexagonal structural segments drawn in the broken lines indicate the inner wall. Bolded solid lines indicate strands in the outer structural segments and bolded broken lines indicate strands in the inner structural segments. A broad layer of dots indicated a sheet of material. A thick solid line on the back side of the device indicated the placement of the optional hinges and the large solid dots connected by a thinner solid line on the front side of the device indicates the location of the longitudinal opening and optional latches.

In FIGS. 5A-5C: 31 is the first end opening, 32 is the second end opening, 33 is the inner wall structural segments, 34 is the outer wall structural segments, 35 is the joining of the inner and outer wall, 36 is the sheet of material sandwiched between the walls, 37 is the strands of material in or between the walls, 38 is a longitudinal opening with optional hinges on one side, and 39 are the latches on the other to facilitate the placement surrounding a biological or other physical structure and to facilitate the removal of the device;

In FIGS. 6A-6C: 41 is the first end opening, 42 is the second end opening, 43 is the inner wall structural segments, 44 is the outer wall structural segments, 45 is the joining of the inner and outer wall, 46 is the sheet of material sandwiched between the walls, 47 is the strands of material in or between the walls, 48 is a longitudinal opening with optional hinges on one side, and 49 are the latches on the other to facilitate the placement surrounding a biological or other physical structure and to facilitate the removal of the device.

In FIGS. 7A-7C: 51 is the first end opening, 52 is the second end opening, 53 is the inner wall structural segments, 54 is the outer wall structural segments, 55 is the joining of the inner and outer wall, 56 is the sheet of material sandwiched between the walls, 57 is the strands of material in or between the walls, 58 is the longitudinal opening with optional hinges on one side, and 59 are the latches on the other to facilitate the placement surrounding a biological or other physical structure and to facilitate the removal of the device; and

In FIGS. 8A-8F: 61 is the first end opening, 62 is the second end opening, 63 is the inner wall structural segments, 64 is the outer wall structural segments, 65 is the joining of the inner and outer wall on the first end, 66 is the joining of the inner and outer wall on the second end, 67 is the sheet of material sandwiched between the walls, 68 is the strands of material in or between the walls, 69 is the sheet of material surrounding the single wall of the device, 70 a longitudinal opening with optional hinges on one side, and 71 are the latches on the other to facilitate the placement surrounding a biological or other physical structure and to facilitate the removal of the device.

### DETAILED DESCRIPTION OF PREFERRED EMBODIMENTS

A preferred embodiment of the present invention is a double-wall device that forms a tube. The double-wall tubular device may be: (1) formed by two tubular structures placed one inside the other with or without a seam or seams at either or both ends (FIG. 1A), (2) woven to be a seamless double wall tube at both ends (FIG. 2A), (3) woven to be a seamless double wall tube on one end and a seam on the other end (FIG. 3A); or (4) a double wall tube on the first end and the second end connected by a single wall middle segment (FIG. 4A)

This double-wall construction facilitates removal and the easy incorporation of other useful materials between the two walls. These materials can deliver drug substances, supplements, minerals, biological materials such as stem cells, or a combination thereof. Additionally, these materials can prevent adhesion, facilitate flow through the device and/or facilitate the device recovery.

In the single sheet folded configuration or the woven tube configuration the leading edge of the device forms a fold that can be shaped to cause the fluid in the tube to increase velocity and maintain laminar flow. This increase in velocity can overcome the fluid turbulence noted when a previously flexible tube is caused to be ridged due to injury, repair or placement of a device which does not respond to variable pressure caused by beating contractile pumps. Additionally this increased velocity through the device will help maintain patency. The inside diameter of this configuration can be adjusted throughout its length to help maintain laminar flow by use of a segmented balloon catheter and a Doppler device to monitor turbulence. The final adjustment would result in the least turbulent flow through the device.

Additionally, the device can be removed with a special catheter equipped with a guarded treble hook canula. The structure of the device causes it to reduce its diameter when the hooks are engaged and a pulling and twisting pressure are applied. Removal is facilitated by pulling the device into the catheter that has been advanced to the first end of the device. Then the guarded treble hook canula is advanced out the end of the catheter and the hooks are engaged in the inner wall structural segments near the second end of the device. The device is then drawn slowly and carefully into the catheter and the catheter removed from the tubular structure.

Another preferred embodiment of the present invention is a double-wall device that forms a tube. The double-wall expandable device may be: (1) formed by two tubular structures placed one inside the other with or without a seam or seams at either or both ends (FIG. 5A), (2) woven to be a seamless double wall tube at both ends (FIG. 6A), (3) woven to be a seamless double wall tube on one end and a seam on the other end (FIG. 7A); or (4) a double wall tube on the first end and the second end connected by a single wall middle segment (FIG. 8A)

Additionally, the device is designed with the length of the device capable of being opened and closed to facilitate placement surrounding the treated structure to form a splint to offer protection, facilitate healing and deliver drugs or biological materials to the treated structure. This capability of being openable and closable can be accomplished with the use of latches, hinges, or memory metals such as nitinol. The latches can be mechanical, magnetic or any other method by which the two halves of the device can be secured together. The hinge may be mechanical with hinge-pins, a curved memory hinge, or a nano-scale ductile hinge. The combination of hinges and latches can be optional and variable depending on the size and use of the device.

In one aspect, when opened, one embodiment of the multi-wall present invention has two C-shaped multi-wall pieces which could be optionally be joined by one or more hinges. One of the types of hinges that can be used in this device have been described in U.S. Pat. Nos. 6,241,762; 6,290,673; 6,293,967; 6,527,799; 6,562,065; and 6,764,507; the entire content of each of which is hereby incorporated by reference. When closed, the expandable multi-wall device takes the form of a tube. The tube can be optionally held together by one or more latched holding each of the two C-shaped multi-walls. Multi-wall materials having memory can close and remain closed due to the memory of the materials

Medical grade stainless steel is the most common material from which the multi-wall expandable device can be made. Alternate materials that can be used for the multi-wall expandable device include, but are not limited to, nitinol, titanium, tantalum, cobalt-based alloys, bioresorbable materials, ceramics, plastics, composites, and polymers. Bioabsorbable polymers that could be used for the device include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Biostable polymers such as polyurethanes, silicones, and polyesters could also be used the multi-wall expandable device of the present invention. Other polymers could likewise be used if they can be dissolved and cured or polymerized on the device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; carbon structures such as carbon nano-tubes; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The device can be used to deliver specific drugs for a medical condition to a specific site. FIGS. 1B-1C, 2B-2C, 3B-3C, 4B-4F, 11B-11C, 12B-12C, 13B-13C and 14B-14F illustrate a drug delivery sheet or strands sandwiched between the two layers of the device structural material that can be impregnated with one or more drugs. The term “drug, ” “therapeutic” and/or “bioactive agent” as used herein means any compound intended for use in animals having a desired effect. Non-limiting examples include anticoagulants, such as an RGD peptide-containing compound, heparin, antithrombin compounds, platelet receptor antagonists, anti-thrombin antibodies, anti-platelet receptor antibodies, aspirin, protaglandin inhibitors, platelet inhibitors, or tick anti-platelet peptide. Other classes of drugs includes vascular cell antiproliferative agents, such as a growth factor inhibitor, growth factor receptor antagonists, transcriptional repressor or translational repressor, antisense DNA, antisense RNA, replication inhibitor, inhibitory antibodies, antibodies directed against growth factors, cytotoxic agents, cytoskeleton inhibitors, peroxisome proliferator-activated receptor gamma (PPAR.gamma.) agonists, molecular chaperone inhibitors and bifunctional molecules. The drug can also include cholesterol-lowering agents, vasodilating agents, and agents which interfere with endogenous vasoactive mechanisms. Other examples of drugs can include anti-inflammatory agents, anti-platelet or fibrinolytic agents, anti-neoplastic agents, anti-allergic agents, anti-rejection agents, metalloprotease inhibitors, anti-microbial or anti-bacterial or anti-viral agents, hormones, vasoactive substances, anti-invasive factors, anti-cancer drugs, antibodies and lymphokines, anti-angiogenic agents, radioactive agents and gene therapy drugs, among others.

Specific non-limiting examples of drugs that fall under one or more of the above categories include paclitaxel, docetaxel and derivatives, epothilones, nitric oxide release agents, heparin, aspirin, coumadin, D-phenylalanyl-prolyl-arginine chloromethylketone (PPACK), hirudin, polypeptide from angiostatin and endostatin, benzoquinone ansamycins including geldanamycin, herbimycin and macbecin, methotrexate, 5-fluorouracil, estradiol, P-selectin Glycoprotein ligand-1 chimera, abciximab, exochelin, eleutherobin and sarcodictyin, fludarabine, sirolimus, rapamycin, tetrazole-containing immunosuppressant macrolide antibiotics (for example Abbott Laboratories ABT-578. See, for example U.S. Pat. No. 6,015,815. Specifically, Examples 1, 1A and 2 for synthesis and claims 1, 2 and 3 for structures, all of which are incorporated herein by reference), certican, Sulindac, tranilast, thiazolidinediones including rosiglitazone, troglitazone, pioglitazone, darglitazone and englitazone, tetracycline antibiotics (tetracyclines), VEGF, transforming growth factor (TGF)-beta, insulin-like growth factor (IGF), platelet derived growth factor (PDGF), fibroblast growth factor (FGF), RGD peptide, estrogens including 17 beta-estradiol, metalloprotease inhibitors and beta or gamma ray emitter (radioactive) agents.

As shown in FIGS. 1B-1C, 2B-2C, 3B-3C, 4B-4F 11B-11C, 12B-12C, 13B-13C and 14B-14F, the film or sheet material between the inner and outer layer of the device does not allow the film or sheet material to escape or be lost during the procedure. This film or sheet material can be used to prevent adhesion, to prevent leakage, or to act as a drug delivery material. In the drug delivery usage, generally, the multiple-wall expandable device is configured to provide a low profile that facilitates device delivery (e.g., via a catheter) and deployment/expansion within the tubular structure of the patient. As used herein a “film, sheet or strand” may be either woven from individual polymeric stands, extruded as a single intact sheet or tube, as in the case of polytetrafluoroethylene (PTFE AKA Teflon.RTM.) and similar polymers or milled from a solid polymer into a sheet.

In an alternate embodiment of the invention, the drug delivery film, sheet, or strands are fabricated from an elastic-type material having expansion and compression characteristics similar to those of the device. In some instances, when the fibers or elements comprising the sheath material expand to accommodate the shape of the implanted device, not only do the fibers elongate but the spaces or pores between the fibers also increase is size. As such, fluids such as blood, systemically-delivered drugs, activator agents, and other fluids known to those skilled in the art flow through the lumen and pores of the device saturating both the device and the target tissue. This device configuration is thought to provide improved fluid flow through the walls of the device and to the tissue target site, which may also produce enhanced therapeutic and diagnostic capabilities.

In an alternate embodiment of the invention, more than one sheet or strand may be applied to a device. Although only two sheaths are illustrated, it is understood that multiple sheaths may be used and are included within the scope of the claimed invention.

As is evident from the previously described embodiments, the drug-loaded sheets or strands are secured between the layers of the multi-wall expandable device. In an alternate embodiment (not shown), the sheets may be secured to the devices via hooks, adhesives, welds, chemical bonds, stitches. In general, the sheets or strands should be sufficiently secured onto the devices to prevent device migration within or dislodgement from the target site within the lumen of the tubular structure treated.

In general, the sheets or strands of the material are woven onto the devices in order to securely attach the material onto the device in a manner that does not interfere with device deployment. The drug delivery sheet(s) or strand(s) of the present invention may be fabricated from one or more materials that are biocompatible, non-toxic and capable of delivering drugs, supplements or minerals to a target site. The sheet or strand material and its structure should also be configured to allow fluids/blood to flow through the wall of the sheath/strand. This design feature not only allows fluids to contact the tissue areas adjacent the device, but also prevents side branch occlusion in the event that the device(s) is deployed at or near a vessel side branch.

Examples of film, sheet, or strand materials that may be used with the device or devices of the present invention include, but are not limited to, resorbable polymers, synthetic polymers, natural polymers including fibrin, fibrinogens, starches and collagens, polyglycolic acid (PGA), poly(L-lactic acid) (PLLA), polydioxanone (PDS), poly(D,L-lactic acid) (PDLLA), polycaprolactone, poly(lactide-co-glycolide), poly(hydroxybutyrate), poly(hydroxybutyrate-co-valerate), polyorthoester, polyanhydride, poly(glycolic acid-co-trimethylene carbonate), polyphosphoester, polyphosphoester urethane, poly(amino acids), cyanoacrylates, poly(trimethylene carbonate), poly(immunocarbonate), copoly(ether-esters) (e.g., PEO/PLA), polyalkylene oxalates, polyphosphazenes, copolymers, tyrosine-derived polycarbonates, carbon structures and compounds such as carbon nano-tubes; tricalcium phosphates, celluloses, hyaluronic acids, gels, proteins, allografts, hydrogels, PTFE (Polytetrafluoroethylene), Vicryl.RTM. (manufactured by Ethicon, New Jersey) Prolenee (manufactured by Ethicon, New Jersey), Mersilene.RTM. (manufactured by Ethicon, New Jersey), polyethylene fiber, and GORE-TEX.RTM. (manufactured by W. L. Gore & Associates, Arizona). In addition, biostable polymers with a relatively low chronic tissue response such as polyurethanes, silicones, polyesters, polyolefins, polyisobutylene and ethylene-alphaolefin copolymers, acrylic polymers and copolymers, vinyl halide polymers and copolymers, such as polyvinyl chloride; polyvinyl ethers, such as polyvinyl methyl ether, polyvinylidene halides, such as polyvinylidene fluoride and polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose and other materials, including combinations thereof, known by those skilled in the art may also be used and are also included within the scope of the claimed invention.

A unique application of this device is the delivery of stem cells in a culture matrix via catheter to tissues to be implanted and revitalized. A double wall device with a matrix between the walls suitable for the adherence of cells will be placed in a culture of stem cells. As these cells colonize the matrix, they will develop connections between adjoining cells on the matrix. When the device is colonized and the stem cells are stable, the device is then transferred to the implantation site via catheter and the device is allowed to remain for a few days. During this time the cells will migrate from the device matrix and colonize the adjoining tissues. Additionally they will maintain their connections established in culture to the adjoining cells as they migrate into the tissues. This capability is especially important if the stem cells are being used to repair cardiac tissues o nerve tissues or any other tissue where cell-cell communications are important. The transport device can then be removed if required.

Other treatment or diagnostic procedures, or both, utilizing materials of various combinations of sheets, sheet designs, strands, strand designs, drugs, release agents, and medical procedures with the multi-wall expandable device of the present invention, not disclosed herein but known to those skilled in the art, are also included within the scope of the claimed invention. As such, the multi-wall expandable device of the present invention can provide for structural support, adhesion prevention, low pressure tubular system support, localized drug delivery, long-term treatment and/or diagnostic capabilities. In addition, the multi-wall expandable device of the present invention as referenced above provide increased efficiency, therapeutic or diagnostic effectiveness, cost-effectiveness and user convenience.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

### REFERENCES CITED

The following references, to the extent that they provide exemplary procedural or other details supplementary to those set forth herein, are specifically incorporated herein by reference.

### U.S. PATENT DOCUMENTS:

U.S. Patent Application Publication. Pub. No. US 2004/0236415 A1;Pub. Date Nov. 25, 2004, entitled “Medical Devices Having Drug Releasing Polymer Reservoirs,” with Richard Thomas listed as inventor.

U.S. Pat. No. 6,699,275 B1, entitled “Stent and Delivery System,” with Mark B. Knudson, John P. Sopp, and Timothy R. Conrad as inventors.

U.S. Pat. No. 6,716,225 B2, entitled “Implant devices for Nerve Repair,” with Shu-Tung Li, Debbie Yuen as inventors.

U.S. Pat. No. 6,241,762 B1, entitled “Expandable Medical Device with Ductile Hinges” with John F. Shanley as inventor.

U.S. Pat. No. 6,290,673 B1, entitled “Expandable Medical Device Delivery System and Method” with John F. Shanley as inventor.

U.S. Pat. No. 6,293,967 B1, entitled “Expandable Medical Device with Ductile Hinges” with John F. Shanley as inventor.

U.S. Pat. No. 6,527,799 B2, entitled “Expandable Medical Device with Ductile Hinges” with John F. Shanley as inventor.

U.S. Pat. No. 6,562,065 B1, entitled “Expandable Medical Device with Beneficial Agent Delivery Mechanism” with John F. Shanley as inventor.

U.S. Pat. No. 6,764,507 B2, entitled “Expandable Medical Device with Improved Spatial Distribution” with John F. Shanley, Neal L. Eigler, and Elazer R. Edelman as inventors.

### REFERENCES:

KUTRYK, Drug-eluting stents for the treatment of coronary artery disease Part 4: New results from clinical trials and future directs, Cardiology Rounds, Vol. 8, lss. 10 Dec. 2003
DEGERTEKIN, Very long sirolimus-eluting stent implantation for de vovo coronary lesions, Am J. Cardiol., 2004 Apr. 1, 2004 (7):826-9

What is claimed:

1. A multi-wall expandable device comprising:
a plurality of radially expandable tubular members, each tubular member axially extending along a longitudinal axis of the multi-wall expandable device, and each tubular member having first and second opposing ends, wherein adjacent tubular members are connected at least at one end to form a seamless joint.

2. The multi-wall expandable device of claim 1, wherein the tubular members are solid materials, perforated materials, strands, or a combination thereof.

3. The multi-wall expandable device of claim 1, wherein the seamless joint comprises a folding.

4. The multi-wall expandable device of claim 1, wherein the tubular members are made of stainless steel, nitinol, titanium, tantalum, cobalt-based alloys, bioresorbable materials, ceramics, plastics, composites, or polymers.

5. The multi-wall expandable device of claim 1, further comprising a therapeutic-delivery material on the outside of the multi-wall expandable device or interdisposed between woven tubular members.

6. The multi-wall expandable device of claim 5, wherein the therapeutic-delivery material is cable of delivering an anticoagulant, an anti-inflammatory agent, an anti-platelet, an anti-angiogenic agent, a fibrinolytic agent, a cell, a stem cell, a protein, an DNA, or a combination thereof.

7. The multi-wall expandable device of claim 5, wherein the therapeutic-delivery material is in the form of a film, a woven material, or a combination thereof.

8. A multi-wall expandable device comprising:
at least two radially expandable tubular members, each tubular member axially extending along a longitudinal axis of the multi-wall expandable device, and each tubular member having first and second opposing ends, wherein the two tubular members are connected at least at one end to form a seamless joint, and the two tubular members are openable and closable along the longitudinal axis of the multi-wall expandable device via a latch, a hinge, or a memory metal.

9. The multi-wall expandable device of claim 8, wherein the tubular members are solid materials, perforated materials, strands, or a combination thereof.

10. The multi-wall expandable device of claim 8, wherein the seamless joint comprises a folding.

11. The multi-wall expandable device of claim 8, wherein the tubular members are made of stainless steel, nitinol, titanium, tantalum, cobalt-based alloys, bioresorbable materials, ceramics, plastics, composites, or polymers.

12. The multi-wall expandable device of claim 8, further comprising a therapeutic-delivery material on the outside of the multi-wall expandable device or interdisposed between tubular members.

13. The multi-wall expandable device of claim 12, wherein the therapeutic-delivery material is cable of delivering an anticoagulant, an anti-inflammatory agent, an anti-platelet, an anti-angiogenic agent, a fibrinolytic agent, a protein, an DNA, or a combination thereof.

14. The multi-wall expandable device of claim 10, wherein the therapeutic-delivery material is in the form of a film, a woven material, or a combination thereof.

15. A double-wall expandable device comprising:
one inner and one outer radially expandable tubular members, the inner and the outer tubular member axially extending about a longitudinal axis of the double-wall expandable device, and each inner and outer tubular member having first and second opposing ends, wherein the two tubular members are connected at least at one end to form a seamless joint.

16. The multi-wall expandable device of claim 15, wherein the tubular members are solid materials, perforated materials, strands, or a combination thereof.

17. The double-wall expandable device of claim 15, wherein the seamless joint comprises a folding.

18. The double-wall expandable device of claim 13, wherein the tubular members are made of stainless steel, nitinol, titanium, tantalum, cobalt-based alloys, bioresorbable materials, ceramics, plastics, composites, or polymers.

19. The double-wall expandable device of claim 13, further comprising an therapeutic-delivery material on the outside of the multi-wall expandable device or interdisposed between woven tubular members.

20. The double-wall expandable device of claim 19, wherein the therapeutic-delivery material is cable of delivering an anticoagulant, an anti-inflammatory agent, an anti-platelet, an anti-angiogenic agent, a fibrinolytic agent, a cell, a stem cell, a protein, an DNA, or a combination thereof.

21. The double-wall expandable device of claim 20, wherein the therapeutic-delivery material is in the form of a film, a woven material, or a combination thereof.

22. A multi-wall expandable device comprising:
one inner and one outer radially expandable tubular members, the inner and the outer tubular member axially extending about a longitudinal axis of the multi-wall expandable device, and each inner and outer tubular member having first and second opposing ends.

23. The multi-wall expandable device of claim 22, wherein the tubular members are solid materials, perforated materials, strands, or a combination thereof.

24. The multi-wall expandable device of claim 22, wherein the tubular members are made of stainless steel, nitinol, titanium, tantalum, cobalt-based alloys, bioresorbable materials, ceramics, plastics, composites, or polymers.

25. The multi-wall expandable device of claim 22, further comprising an therapeutic-delivery material on the outside of the multi-wall expandable device or interdisposed between woven tubular members.

26. The multi-wall expandable device of claim 25, wherein the therapeutic-delivery material is cable of delivering an anticoagulant, an anti-inflammatory agent, an anti-platelet, an anti-angiogenic agent, a fibrinolytic agent, a cell, a stem cell, a protein, an DNA, or a combination thereof.

27. The multi-wall expandable device of claim 25, wherein the therapeutic-delivery material is in the form of a film, a woven material, or a combination thereof.
